(19) United States
(12) Patent Application Publication
(10) Pub. No.: US 2023/0347144 A1
(43) Pub. Date: Nov. 2, 2023

(54) BIOELECTRIC OPG TREATMENT OF CANCER

(71) Applicant: Leonhardt Ventures LLC, Mission Viejo, CA (US)

(72) Inventors: Howard J. Leonhardt, Mission Viejo, CA (US); Jorge Genovese, Irvine, CA (US)

(21) Appl. No.: 18/347,073

(22) Filed: Jul. 5, 2023

Related U.S. Application Data
(63) Continuation-in-part of application No. 15/812,760, filed on Nov. 14, 2017, now Pat. No. 10,960,206.
Application No. 15/812,760 is a continuation-in-part of application No. 15/460,129, filed on Mar. 15, 2017, now Pat. No. 10,646,644.
Application No. 15/460,129 is a continuation-in-part of application No. 15/471,954, filed on Mar. 28, 2017, now Pat. No. 10,695,563.
(60) Provisional application No. 62/308,702, filed on Mar. 15, 2016.
(60) Provisional application No. 62/363,012, filed on Jul. 15, 2016.
(60) Provisional application No. 62/364,472, filed on Jul. 20, 2016.
(60) Provisional application No. 62/375,271, filed on Aug. 15, 2016.
(60) Provisional application No. 62/385,124, filed on Sep. 8, 2016.
(60) Provisional application No. 62/454,521, filed on Feb. 3, 2017.
(60) Provisional application No. 62/352,930, filed on Jun. 21, 2016.
(60) Provisional application No. 62/314,240, filed on Mar. 28, 2016.

(51) Int. Cl. A61N 1/20

(52) U.S. Cl. CPC A61N 1/205

(57) ABSTRACT

Age is an important predictor of neuromuscular recovery after peripheral nerve injury. Insulin-like growth factor 1 (IGF-1) is a potent neurotrophic factor that is known to decline with increasing age. The purpose of this study was to determine if locally delivered IGF-1 would improve nerve regeneration and neuromuscular recovery in aged animals. Young and aged rats underwent nerve transection and repair with either saline or IGF-1 continuously delivered to the site of the nerve repair. After 3 months, nerve regeneration and neuromuscular junction morphology were assessed. In both young and aged animals, IGF-1 significantly improved axon number, diameter, and density. IGF-1 also significantly increased myelination and Schwann cell activity and preserved the morphology of the postsynaptic neuromuscular junction (NMJ), These results show that aged regenerating nerve is sensitive to IGF-1 treatment. Muscle Nerve, 2009

### CROSS-REFERENCE TO RELATED APPLICATIONS

This application is a continuation of U.S. patent application Ser. No. 16/913,964, filed Jun. 26, 2020, U.S. Pat. No. 11,691,007, Jul. 4, 2023, which is a continuation-in-part of U.S. patent application Ser. No. 16/137,035, filed on Sep. 20, 2018 (US 20190030330 (Jan. 31, 2019)), which is a continuation-in-part of U.S. patent application Ser. No. 15/460,129, filed on Mar. 15, 2017 (U.S. 2017/0266371A1, Sep. 21, 2017), which itself claims the benefit under 35 USC § 119 of U.S. Provisional Patent Application Ser. No. 62/308,702, filed Mar. 15, 2016, U.S. Provisional Patent Application Ser. No. 62/363,012, filed Jul. 15, 2016, U.S. Provisional Patent Application Ser. No. 62/364,472, filed Jul. 20, 2016, U.S. Provisional Patent Application Ser. No. 62/375,271, filed Aug. 15, 2016, U.S. Provisional Patent Application Ser. No. 62/385,124, filed Sep. 8, 2016, U.S. Provisional Patent Application Ser. No. 62/454,521, filed Feb. 3, 2017, and U.S. Provisional Patent Application Ser. No. 62/352,930, filed Jun. 21, 2016, the disclosure of each of which is incorporated herein in its entirety by this reference.

This application is also a continuation-in-part of application Ser. No. 15/812,760, filed Nov. 14, 2017 (US 2018/0064935 A1, Mar. 8, 2018), which is a continuation-in-part of U.S. Ser. No. 15/460,129, filed on Mar. 15, 2017 (US 2017/0266371A1, Sep. 21, 2017), which itself claims the benefit under 35 USC § 119 of U.S. Provisional Patent Application Ser. No. 62/308,702, filed Mar. 15, 2016, U.S. Provisional Patent Application Ser. No. 62/363,012, filed Jul. 15, 2016, U.S. Provisional Patent Application Ser. No. 62/364,472, filed Jul. 20, 2016, U.S. Provisional Patent Application Ser. No. 62/375,271, filed Aug. 15, 2016, U.S. Provisional Patent Application Ser. No. 62/385,124, filed Sep. 8, 2016, U.S. Provisional Patent Application Ser. No. 62/454,521, filed Feb. 3, 2017, and U.S. Provisional Patent Application Ser. No. 62/352,930, filed Jun. 21, 2016, the disclosure of each of which is incorporated herein in its entirety by this reference.

This application is also a continuation in part of U.S. application Ser. No. 15/471,954, filed Mar. 28, 2017, which claims the benefit under 35 USC § 119 of U.S. Provisional Patent Application Ser. No. 62/314,240, filed Mar. 28, 2016, the disclosure of each which is incorporated herein in its entirety by this reference.

### TECHNICAL FIELD

The application relates generally to the field of medical devices and associated treatments, and to precise bioelectrical stimulation of a subject's tissue, potentially augmented with the administration of a composition comprising, among other things, stem cells and nutrients, useful to stimulate and treat the subject, the subject's tissue(s), the subject's organ(s), and/or the subject's cells. More specifically described is a bioelectric therapy protocol for cancer, especially multiple myeloma or bone cancer treatment.

### BACKGROUND

Multiple myeloma is a cancer in which plasma cells grow out of control. Healthy plasma cells come from bone marrow and play an important role in the immune system. With multiple myeloma however, the plasma cells create a buildup of an abnormal protein in bones and blood, which can lead to symptoms such as bone pain, anemia, and infections. Multiple myeloma is rarely cured, so treatments typically focus on relieving symptoms, reaching remission, and helping the patient live longer. Existing treatments include targeted therapies and stem cell transplants.

As described by S. Goranov (2004) infra, multiple myeloma induces considerable imbalance in the osteoprotegerin (“OPG”), receptor activator of nuclear factor KB ligand (“RANKL”) and RANK system. Signaling pathways for differentiation and proliferation of an osteoclastic line activates RANKL's binding to RANK on the surface of osteoclastic precursors in the presence of m-CSF. OPG is a decoy circulating receptor for RANKL that blocks RANKL's binding to RANK. At least three mechanisms exist by which myeloma cells affect the RANK/RANKL/OPG system. First, the adhesion between myeloma/stromal cells and the osteoblastic precursors stimulates the system by increasing the production of RANKL. Second, some myeloma lines produce membrane-bound or free RANKL. Third, the normal and mutated plasma cells bind, degrade, and block OPG production from stromal cells. The first results from the application of a synthetic analogue of OPG, as well as by RANKL antagonists or RANK inhibitors, which show decrease in the number of osteoclasts, osteolytic lesions, and M-gradient.

A number of published studies have linked a role of RANK/RANKL signaling in patients with multiple myeloma, who have increased serum levels of soluble RANKL and an imbalance in RANKL and OPG. Current therapies for patients with multiple myeloma demonstrate that RANKL may be released by tumor cells or OPG osteoprogenitor cells. This RANKL overexpression in simple terms eats away and destroys bone in multiple myeloma patients.

Previous research has focused on injecting or infusing OPG for inhibiting RANKL over expression and thus reducing or stopping bone degradation.

### BRIEF SUMMARY

Described is the application is a bioelectric signal or signals to upregulate expression of OPG at levels in a multiple myeloma patient that inhibit over-expression of RANKL and thus also inhibit bone degradation in the multiple myeloma patient. This treatment may be combined with other bioelectric signal treatment protocols (such as those described in the incorporated US 2018/0064935 A1) in an attempt to control cancer spread in the patient.

Disclosed are means for upregulating the expression of OPG to inhibit RANKL over-expression in a subject. Typically, the subject will have been diagnosed with cancer, breast cancer, bone cancer, lung cancer, osteoporosis, multiple myeloma, and a combination of any thereof. Bioelectric signaling is utilized to deliver an effective and safe option for patients with fewer toxic side effects than may be experienced with chemo and radiation therapies.

RANKL and its decoy receptor OPG are regulators of bone homeostasis and have been implicated in the development and progression of, for example, breast cancer. Rachner et al. (2019) infra demonstrated that high levels of OPG were an independent prognostic marker for breast cancer—specific survival (“BCSS”), while relatively high levels of RANKL indicated improved BCSS in disseminated tumor cell (“DTC”)-negative patients. RANKL levels were increased in DTC-positive patients and in patients who later developed bone metastases.

The described bioelectric signaling technology targets specific cancers such as multiple myeloma. The technology is based on bioelectric controlled expression of OPG to inhibit RANKL over production, which otherwise eats away at bone in multiple myeloma patients and contributes to the spread of other cancers such as lung cancer(s).

Described is a bioelectric stimulator programmed to produce a bioelectric signal or bioelectric signals that stimulate(s) target tissue in a subject, wherein the bioelectric signal(s) comprise(s) a signal of, within 15%, 4.0 milliVolt (mV), 2,000 Hz, square wave, and/or a signal of from 3 mV to 5 mV at a frequency range of 1 to 3 MHz, and a duration range of 30 to 40 mW/cm2 for a minimum of 20 to 45 minutes. Such bioelectric signals, applied to a human cell or tissue, increase the expression of OPG by the cell or tissue.

In certain embodiments, the bioelectric stimulator of claim 1, wherein the bioelectric stimulator is further programmed to produce a signal of 3.0 mV, 2 Hz, square wave; 3 mV at 2/100 Hz alternating frequency with current of 3 mA followed by 15 Hz, 1 Gauss EM field, consisting of 5-millisecond bursts with 5-microsecond pulses followed by 200-μs pulse duration at 30 Hz and with current amplitude of 140 mA; and/or a biphasic pulse at 20 Hz, 0.1 V, and a 7.8 ms pulse duration. Such bioelectric signals, applied to a human cell or tissue, increase the expression of RANKL by the cell or tissue. Such expression can be used to offset OPG expression if needed.

Such a bioelectric stimulator may be used to treat a subject diagnosed as suffering from, e.g., a disorder selected from the group consisting of cancer, breast cancer, bone cancer, lung cancer, osteoporosis, and a combination of any thereof, by delivering the bioelectric signal(s) to tissue of the subject. Particularly, a subject suffering who has been diagnosed with multiple myeloma may be treated with the thus programmed bioelectric stimulator.

In certain embodiments, the method of treatment further includes separately delivering to the subject an admixture comprising any combination of the following: stem cells, endothelial progenitor cells, selected exosomes, selected alkaloids, selected anti-inflammatory agents, nutrient hydrogel, organ-specific matrix, selected growth factors, amniotic fluid, placenta fluid, cord blood, and embryonic sourced growth factors and cells.

A system that utilizes the method typically includes:

1. A bioelectric stimulator programmed as described.

2. A micro infusion pump (e.g., a FluidSync™ micropump available from Fluidsynchrony of Pasadena, CA, US), which is programmable and re-fillable and preferably has a low cell damage design. Such a pump preferably includes a refilling silicon septum port or ports and reservoir chambers.

3. A multi-component organ regeneration composition that includes (depending on the application) adipose-derived stem cells, muscle-derived stem cells (when needed for muscle), exosomes, Micro RNAs, nutrient hydrogel, growth factor cocktail, organ-specific matrix, selected alkaloids, and/or selected anti-inflammatory agents.

The pump and stimulator may be associated with (e.g., connected to) the organ to be treated/regenerated with a pacing infusion lead (available from Nanoscribe of Eggenstein-Leopoldshafen, Germany). The interface with the organ varies by organ, e.g., a conductive soft wrap can be used for certain applications.

Typical treatment times for the OPG bioelectric signals are 20 minutes 2 or 3 times a week (e.g., applied to the patient's jaw). Brain electrodes may also be used to apply the signal. In certain embodiments, the patient is stimulated eight times per day with the OPG bioelectric signal until RANKL is determined to be in balance (which may be determined via an analysis of a blood sample).

Treatment tissues include the jaw, thigh, leg(s), hip, front, and back, or, in certain circumstances, electrode into the bone marrow, which electrode is connected to an implantable stimulator.

### BRIEF DESCRIPTION OF THE DRAWINGS

FIG. 1 depicts a programmed bioelectric stimulator for delivery to cells of a subject via two electrodes. The anode wire/electrode may be placed directly into a tumor or adjacent to the tumor.

FIG. 2 depicts an implantable lead with simulated therapy for liver cancer.

FIG. 3 depicts an interface for use with the system.

FIG. 4 depicts a micropump for use with the system.

FIG. 5 depicts an image of a signal (voltage and frequency) associated with OPG: 4.0 mV, 2,000 Hz, square wave.

FIG. 6 depicts an image of the signal (voltage and frequency) associated with RANKL: 3.0 mV, 2 Hz, square wave.

FIG. 7 depicts a combination bioelectric stimulation and stem cells and growth factors infusion catheter.

FIG. 8 is a close up view of the conductive and infusion corkscrew tip for use with the catheter system of FIG. 7.

### DETAILED DESCRIPTION

In the incorporated US 20190030330, described is, among other things, a multi-modality bioelectric cancer treatment protocol involving certain novel bioelectric signaling sequences for controlling specific actions, which may be adapted to the instant application. The described treatment is customized based on reading the cancer tumors real time with specific signaling sequences delivered customized based on that read. The first wave of bioelectric signals sent to the tumors are designed to jam their communication. The second set of signals are designed to change the surface proteins and electrical surface charge of the tumors to elicit an immune response from the body to kill off the tumor. These signals are specifically designed to elicit a T cell response targeting the proliferating cancer cells. The third wave of treatment is designed to starve a tumor of blood supply by controlled release of anti-angiogenic proteins. The fourth wave of signaling targets the re-programming of cancer cells to re-direct their tumor development pathway. After all of these treatments phases are completed, the bioelectric signaling sequences transition to a promotion of healing and regeneration of the diseased organ including bioelectric inflammation management.

Bioelectric signals may be combined with protein expression and/or release signals, which can be used to, e.g., cut off the blood supply to the cancer tumor. For example, bioelectric signaling can lead to preferably the enhanced expression and/or release of thirteen or more selected proteins (e.g., ones useful for organ regeneration, stopping cell division, enhancing the patient's immunological response, and inhibiting blood supply to the cancer tumor and surrounding tissues). (See, e.g., the bioelectric signals and proteins described in the incorporated US 2018/0064935 A1.)

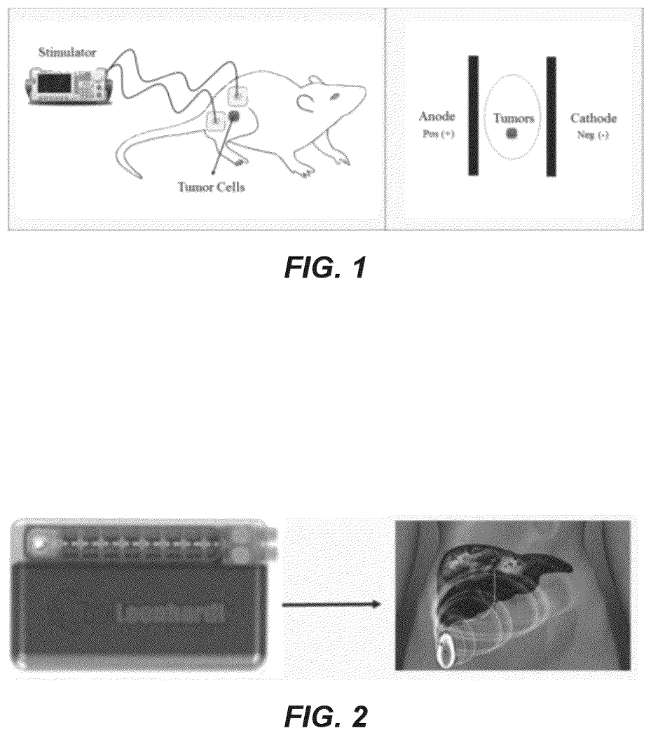

Referring to FIG. 1, depicted is a stimulator for use with treatment of, e.g., a cancer tumor. Preferably, such a device is about the size of two quarters (FIG. 2).

The micro voltage signal generator may be produced utilizing the same techniques to produce a standard heart pacemaker well known to a person of ordinary skill in the art. An exemplary microvoltage generator is available (for experimental purposes from Cal-X Stars Business Accelerator, Inc. DBA Leonhardt's Launchpads or Leonhardt Vineyards LLC DBA Leonhardt Ventures of Salt Lake City, UT, US). The primary difference is the special electrical stimulation signals needed to control, e.g., precise OPG release on demand (which signals are described herein). The leading pacemaker manufacturers are Medtronic, Boston Scientific Guidant, Abbott St. Jude, BioTronik and Sorin Biomedica.

Construction of the electric signal generators and pacemakers are known in the art and can be obtained from OEM suppliers as well as their accompanying chargers and programmers. The electric signal generators are programmed to produce specific signals to lead to specific protein expressions at precisely the right time for, e.g., optimal organ treatment or regeneration.

Micro stimulators may be purchased or constructed in the same manner heart pacemakers have been made since the 1960s. Micro infusion pumps can be purchased or produced similar to how they have been produced for drug, insulin, and pain medication delivery since the 1970s. The programming computer can be a standard laptop computer.

The programming wand customary to wireless programming wands may be used to program heart pacers. A re-charging wand for use herein is preferably similar to the pacemaker re-charging wand developed by Alfred Mann in the early 1970s for recharging externally implantable pacemakers.

Bioelectric stimulation can be done with the described microstimulator, which has a pacing infusion lead with a corkscrew lead placed/attached. The microstimulator is actuated and runs through programmed signals to signal the release of, e.g., OPG. Up-regulation of RANKL and osteoprotegerin (“OPG”) was achieved in bone, tooth, and gum.

In certain embodiments, a signal generator coupled with a voltage amplifier is set to apply electrical stimulation as described herein via needle electrode pair to tumors.

A pacing infusion lead may be constructed or purchased from the same suppliers that build standard heart pacemaker leads. Pacing infusion leads may be purchased from a variety of OEM vendors. The pacing infusion lead may, for example, be a standard one currently used in heart failure pacing studies in combination with drug delivery.

Wireless, single lumen infusion pacing lead(s) or infusion conduction wide array patch(es) may all be used to deliver the regeneration signals and substances to the organ of interest to be treated or they may be used in combination.

An infusion and electrode wide area patch may be constructed by cutting conduction polymer to shape and forming plastic into a flat bag with outlet ports in strategic locations.

The healing process can be accelerated with the use of a micro infusion pump that is filled with various types of stem cells and growth factors and in some cases drugs. Thus, in certain embodiments, the system includes a microinfusion pump (FIG. 4) for continuous or repeat delivery of a liquid composition, which microinfusion pump includes silicon septum ports and associated reservoir chambers connected to the bioelectric stimulator microinfusion pump to the tissue with a pacing infusion lead. The pump is preferably programmable and re-fillable with low cell damage design. Refilling may be by silicon septum ports and reservoir chambers.

In certain embodiments, the system includes an organ regeneration composition comprising, for example, adipose-derived stem cells, bone marrow-derived stem cells, muscle-derived stem cells (e.g., when needed for muscle), exosomes, MicroRNAs, nutrient hydrogel, growth factor cocktail, organ-specific matrix, selected alkaloids, and/or selected anti-inflammatory agents.

An organ-specific matrix is a composition comprising cells of an organ which is to be treated. The organ-specific matrix is believed to aid in stem cell differentiation, but in any event is found to be useful in the composition. It has been found that for the multicomponent composition, cells plus selected growth factors are better than just cells alone. See, e.g., Procházka et al. “Therapeutic Potential of Adipose-Derived Therapeutic Factor Concentrate for Treating Critical Limb Ischemia,” Cell Transplantation, 25(9), pp. 1623-1633(11) (2016) and “Cocktail of Factors from Fat-derived Stem Cells Shows Promise for Critical Limb Ischemia,” world wide web at sciencenewsline.com/news/2016012204520017.html (Jan. 22, 2016), the contents of each of which are incorporated herein by this reference.

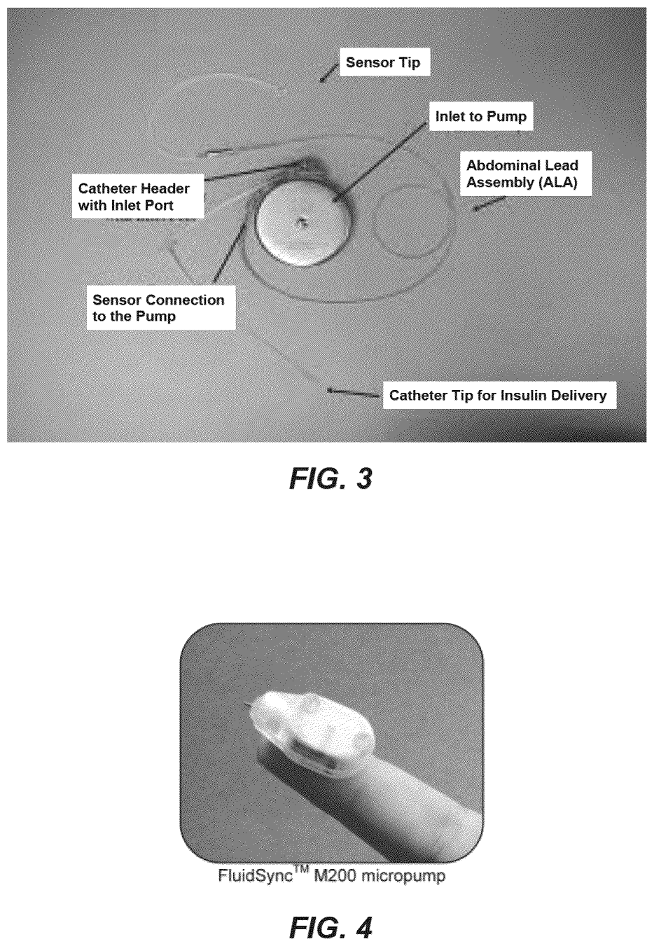

In case of an advanced disease state, a micro infusion pump (e.g., FIGS. 3 and 4) may be used for daily delivery of, e.g., 2 ml of organ regeneration composition (comprised of adipose-derived cells or bone marrow-derived mesenchymal stem cells plus cocktail of growth factors (usually derived from amniotic fluid or placenta), selected Micro RNAs, selected alkaloids, selected anti-inflammatory agents, nutrient hydrogel, organ-specific matrix, selected exosomes). For muscle regeneration, immature myoblasts are included in the composition.

Exosomes represent a specific subset of secreted membrane vesicles, which are relatively homogeneous in size (30-100 nm). Exosomes have been proposed to differ from other membrane vesicles by their size, density, and specific composition of lipids, proteins, and nucleic acids, which reflect its endocytic origin. See Campbell et al. “Electrical stimulation to optimize cardioprotective exosomes from cardiac stem cells” Med Hypotheses. 2016 March; 88:6-9. doi: 10.1016/j.mehy.2015.12.022. Epub 2016 Jan. 11.

One such composition includes adipose-derived cells (or bone marrow derived MSCs or any pluripotent stem cell, such as iPS cells) and growth factor mix which should include SDF-1, IGF-1, EGF, HGF, PDGF, VEGF, eNOS, activin A, activin B, follistatin, relaxin, GDF-10, GDF-11 and tropoelastin plus selected exosomes (miR-146a, miR-294, mES-Exo) plus selected alkaloids (harmine and tetrahydroharmine) plus selected anti-inflammatory factors plus nutrient hydrogel (IGF-1, SDF-1, HGF plus FGF) plus organ-specific matrix. Also, preferably included are amniotic fluid, placenta, or cord blood when available.

The concentration of cells in the compositions is preferably about 50,000,000 cells/ml. The amniotic fluid is preferably as described in Pierce et al. “Collection and characterization of amniotic fluid from scheduled C-section deliveries,” Cell Tissue Bank, DOI 10.1007/s10561-016-9572-7 (Springer, 2012) and is available from Irvine Scientific.

In certain embodiments, a mixed composition is loaded into a micro infusion pump. The pump may be refilled, e.g., weekly to achieve a slow, timed infusion delivery of the composition to the heart scar tissue. Administration of the composition(s) is combined with bioelectric stimulation to control the release of desired proteins. Treatment times for assisting the heart may last 36 months.

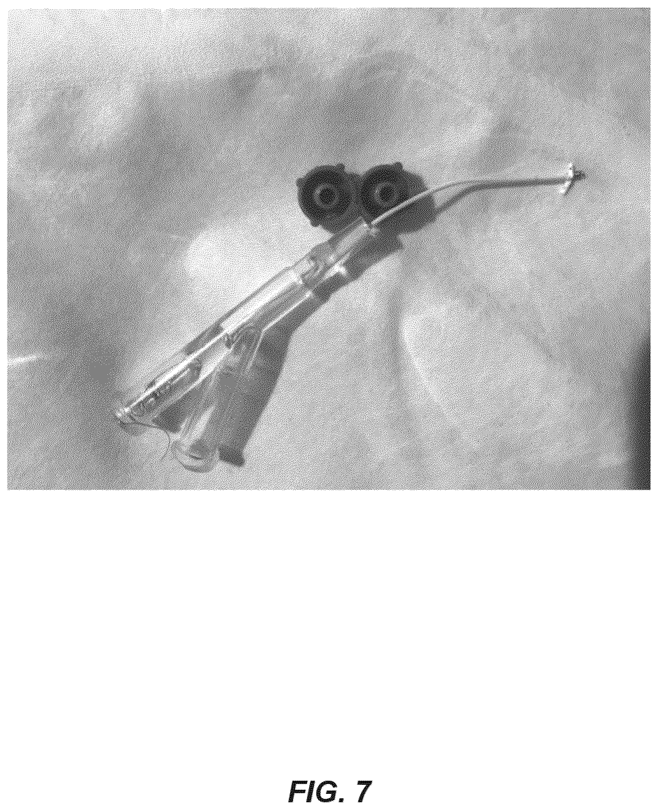

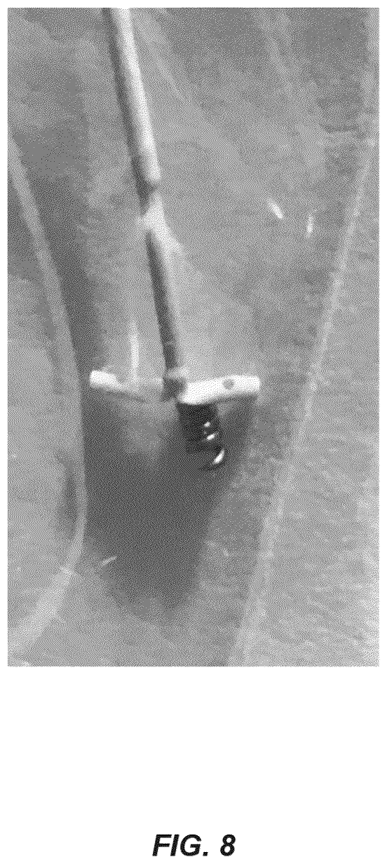

FIG. 7 depicts a combination bioelectric stimulation and stem cell and growth factor(s) infusion catheter usable with the described system.

A corkscrew tip may be of a standard type utilized to secure most heart pacemakers in heart tissue. Wireless delivery of the signal or electro-acupuncture needle delivery is included. FIG. 8 is a close up of the conductive and infusion corkscrew tip for getting deep into target tissue. The tip includes suture tabs for even more secure fixation to the target organ.

Several types of drugs are used to treat multiple myeloma and their respective therapies and dosages are known by those of skill in the art. Such drugs may be taken singly or as a combination. Useful chemotherapeutic agents include bendamustine, cyclophosphamide, doxorubicin, etoposide, liposomal doxorubicin, melphalan, and vincristine. Useful corticosteroids include dexamethasone and prednisone. Useful immunomodulators include lenalidomide, pomalidomide, and thalidomide. Useful monoclonal antibodies daratumumab, elotuzumab, and isatuximab. Panobinostat is a useful Histone Deacetylase (HDAC) inhibitor. Useful proteasome inhibitors include bortezomib, carfilzomib, and ixazomib. Selinexor is a useful nuclear export inhibitor.

When combined with the described OPG bioelectric signal treatment of multiple myeloma, the drug dosage may be less than 20% of a standard dosage for the drug.

A week after treatment, samples can be collected for morphometric evaluation by in-situ hybridization or RT-PCR.

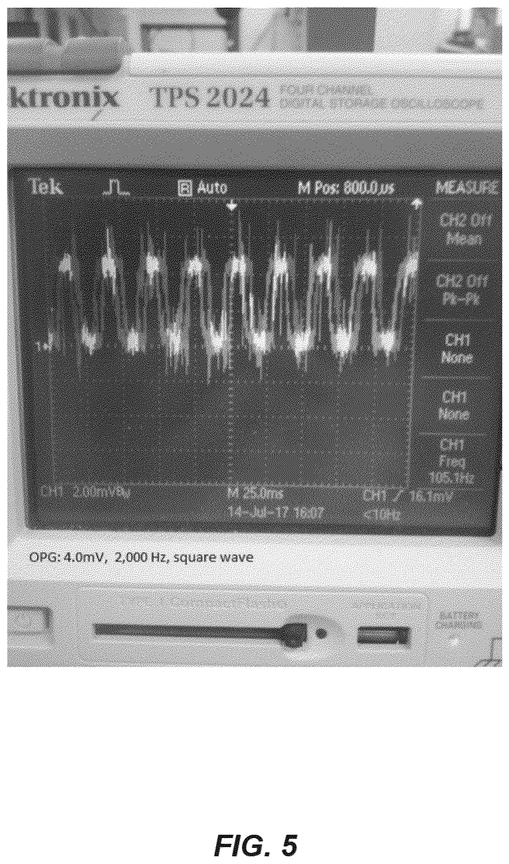

FIGS. 5 & 6 are images of signals with the name, voltage, and frequency of each signal written on each image. The signals are to be further defined in terms of current and frequency, not voltage and frequency as shown. The voltage delivered to the cells will be different for each tissue type, but with current all of the signals can be kept constant regardless of tissue type. The device should have a current driven signal (instead of voltage driven like most other devices).

Specifically, FIG. 5 depicts an image of the signal (voltage and frequency) associated with OPG: 4.0 mV, 2,000 Hz, square wave. Applying this bioelectric signal to gingival cells, eye cells, and rat teeth has increased OPG protein levels in the cells up to 2,300%.

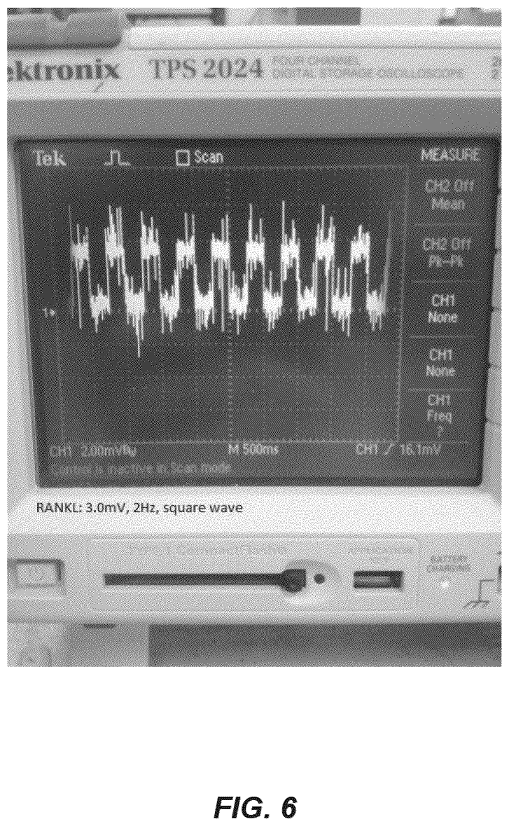

FIG. 6 depicts an image of the signal (voltage and frequency) associated with RANKL: 3.0 mV, 2 Hz, square wave.

The invention is further described with the aid of the following illustrative Examples.

### EXAMPLES

Relationship Between the Components:

The micro voltage signal generator is attached to a pacing infusion lead with, e.g., a corkscrew tip or conductive polymer bandage or patch to the tissue or organ to be treated. An external signal programmer may be used to program the micro voltage signal generator with the proper signals for treatment including the follistatin producing signal. The device battery or other power source (not shown) may be re-chargeable with an external battery charging wand.

In use, the signal generator sends a bioelectric signal to the target tissue, which signal causes the tissue to upregulate expression of OPG. The essential elements are the micro voltage signal generator and the means for delivering the signal to the target tissue.

The signal generator may be external or internal. The transmission of the signal may be wireless, via liquid and/or via wires.

The tissue contact interface may be a patch or bandage or may be via electrodes or leads.

A micro infusion pump may be included in the system for delivering other supportive or therapeutic substances.

The described system upregulates expression of OPG under precise dosing control at safe and comfortable low voltages.

The signal generator programmed with the OPG release signal is directed via a lead, bandage of patch to the target organ tissue in need of muscle repair or build up.

### Example—Treating Cancer Tumors Using Bioelectric Stimulation in Combination with Micro Infusion

Previous cancer treatments failed to address the combination of stopping cell proliferation and blood supply followed by regenerating the damaged tissue or organ.

Cytokine and chemotherapeutic and regenerative treatment for certain cancers may be combined with low intensity, intermediate frequency alternating electric fields that are tuned to release specific beneficial proteins at specific time intervals. More specifically, cell proliferation inhibition and halting blood supply to tumors in the first treatment stage. The bioelectric stimulation treatment may be increased in volume and efficacy by the combination use of an implantable, programmable, re-fillable micro infusion pump that delivers anti-cell proliferation and anti-blood vessel growth proteins as well, if desired, standard cancer treatment drugs such as chemotherapy agents. The second stage of treatment is focused regeneration of cancer damaged tissues back to their most optimal healthy state. The regenerative phase comprises a sequence of recruiting reparative stem cells to the damaged organ by bioelectrically stimulating the release of SDF-1 (stem cell homing factor), followed by a controlled proliferation signal, a controlled blood vessel supply signal (VEGF) and if desired and useful release of Follistatin, tropoelastin, HGF, IGF-1 and Activin. The stimulation cycle causing release of beneficial proteins for regeneration may be upgraded in volume and speed of delivery by the combination use of an implantable, re-fillable, programmable micro infusion pump for delivering a higher quantity of stem cells, nutrient hydrogel, matrix and beneficial tissue and organ regeneration promotion proteins.

Cytokine and Chemotherapeutic and regenerative treatment for certain cancers comprising a combination low intensity, intermediate frequency alternating electric fields that are tuned to release particular beneficial proteins in two stages, stage (1) is stopping cancer spread by halting cell proliferation and halting tumor blood supply and stage (2) regenerating the cancer damaged tissue or organ back to optimal health. In many cases, the resulting cell proliferation inhibition is significantly higher than the inhibition obtained by drug-only regimens of treatment.

A method of killing or inhibiting the growth of cancer cells in a target region followed by regenerating the tissue or organ back to optimal health, the method comprising the steps of:

Stage 1=Stop cancer growth by:

Applying, to the target region, a series of bioelectric signals that damages the cancer cells or inhibits the growth of the cancer cells via stopping cell proliferation and halting blood supply temporarily, but leaves normal cells in the target region substantially unharmed; and

Treating the cancer cells with another anti-cancer regimen via programmable micro pump infusion, wherein the applying step and the treating step are performed simultaneously.

Stage 2=Regeneration of post-cancer tissue or organ by:

Treating the target region with a series of bioelectric signals to recruit stem cells, grow healthy blood vessels and re-grow healthy functional tissues in the previous cancer damaged region.

In such a method, in the applying step, the field may be applied in at least two different directions in an alternating sequence to halt cell proliferation and to stop blood supply to the cancer tumor.

In such a method, the other anti-cancer regimen may comprise treating the cancer cells with an anti-cancer drug. In this method, the drug may comprise at least one drug selected from the group consisting of paclitaxel, doxorubicin cyclophosphamide, and cisplatin. In such a case, the drug dosage may be less than 20% of a standard dosage for the drug.

In such a method, the bioelectric stimulation may release any one of these regenerations of tissue and organ beneficial proteins SDF-1, IGF-1, Activin, HGF, VEGF, Follistatin or tropoelastin and in specific sequences for optimal organ health.

In such a method, all bioelectric regeneration signal may be delivered wirelessly and/or non-invasively.

In such a method, the target cancer may be breast cancer and the target regenerative organ may be breast reconstruction.

### REFERENCES

(The contents of the entirety of each of which is incorporated herein by this reference.)
Ando et al. “RANKL/RANK/OPG: key therapeutic target in bone oncology” Curr Drug Discov Technol. 2008 September; 5(3): 263-268.
Bi et al. “Key Triggers of Osteoclast-Related Diseases and Available Strategies for Targeted Therapies: A Review” Front Med (Lausanne). 2017; 4: 234. doi: 0.3389/fmed.2017.00234.
Buckle et al. “Soluble Rank Ligand Produced by Myeloma Cells Causes Generalised Bone Loss in Multiple Myeloma” PLoS One. 2012; 7(8): e41127. doi: 10.1371/journal.pone.0041127 PMCID: PMC3430669.
Christouls et al. “Pathogenesis and Management of Myeloma Bone Disease” Expert Rev Hematol. 2009; 2(4):385-398.
Fili et al. “Therapeutic implications of osteoprotegerin” Cancer Cell International volume 9, Article number: 26 (2009). doi.org/10.1186/1475-2867-9-26.
S. Goranov “Bone Lesions in Multiple Myeloma—The OPG/RANK-ligand System” Folia Med (Plovdiv). 2004; 46(3): 5-11.
Goswarmi et al. “Osteoprotegerin rich tumor microenvironment: implications in breast cancer” Oncotarget. 2016 Jul. 5; 7(27): 42777-42791.
Holen & Shipman “Role of Osteoprotegerin (OPG) in Cancer” Clin Sci (Lond). 2006 March; 110(3):279-91. doi: 10.1042/CS20050175.
Infante et al. “RANKL/RANK/OPG system beyond bone remodeling: involvement in breast cancer and clinical perspectives” Journal of Experimental & Clinical Cancer Research (2019) 38:12. doi.org/10.1186/s13046-018-1001-2.
Lamoureux et al. “Therapeutic Relevance of Osteoprotegerin Gene Therapy in Osteosarcoma: Blockade of the Vicious Cycle between Tumor Cell Proliferation and Bone Resorption” Cancer Res 1 2007 67(15):7308-7318; DOI: 10.1158/0008-5472.CAN-06-4130.
T. Martin “Historically significant events in the discovery of RANK/RANKL/OPG” World J Orthop. 2013 Oct. 18; 4(4): 186-197. doi: 10.5312/wjo.v4.i4.186.
Liang et al. “Therapeutic effect of low-intensity pulsed ultrasound on temporomandibular joint injury induced by chronic sleep deprivation in rats” Am J Transl Res. 2019; 11(6): 3328-3340.
T. Martin “Historically significant events in the discovery of RANK/RANKL/OPG” World J Orthop. 2013 Oct. 18; 4(4): 186-197. doi: 10.5312/wjo.v4.i4.186.
E. McGrath “OPG/RANKL/RANK Pathway as a Therapeutic Target in Cancer” Journal of Thoracic Oncology, Sep. 2011 6(9): 1468-1473; doi.org/10.10975TO.0b013e318229421f.
D. Novack “Inflammatory osteoclasts, a different breed of bone eaters?” Arthritis Rheumatol. 2016 December; 68(12): 2834-2836. doi: 10.1002/art.39835.
Oyajobi, B. “Multiple myeloma/hypercalcemia” Arthritis Research & Therapy volume 9, Article number: S4 (2007).
Oranger et al. “Cellular Mechanisms of Multiple Myeloma Bone Disease” Clinical and Developmental Immunology Volume 2013, Article ID 289458, 11 pages dx.doi.org/10.1155/2013/289458.
Rachner et al. “Prognostic Value of RANKL/OPG Serum Levels and Disseminated Tumor Cells in Nonmetastatic Breast Cancer” Clin Cancer Res Feb. 15, 2019 (25) (4) 1369-1378; DOI: 10.1158/1078-0432.CCR-18-2482.
Raje et al. “Role of the RANK/RANKL Pathway in Multiple Myeloma” Clin Cancer Res 2019 25(1):12-20; DOI: 10.1158/1078-0432.CCR-18-1537.
Sisay et al. “The RANK/RANKL/OPG system in tumorigenesis and metastasis of cancer stem cell: potential targets for anticancer therapy” Onco Targets Ther. 2017; 10: 3801-3810.
Van Dam et al. “RANK/RANKL signaling inhibition may improve the effectiveness of checkpoint blockade in cancer treatment” Critical Reviews in Oncology/Hematology Volume 133, January 2019, Pages 85-91.
Zdzisińska B, et al. “RANK/RANKL i OPG w szpiczaku plazmocytowym [The role of RANK/RANKL and OPG in multiple myeloma]” Postepy Hig Med Dosw (Online). 2006; 60:471-482.

What is claimed is:

1. A bioelectric stimulator programmed to produce at least one bioelectric signal that stimulate(s) target tissue of a subject to increase production of osteoprotegerin (OPG), wherein the bioelectric signal(s) comprise(s):
from 3 millivolts to 5 millivolts at a frequency range of 1 to 3 MHz, and a duration range of 30 to 40 mW/cm2.

2. A method of upregulating production of osteoprotegerin (OPG) in a living cell, the method comprising:
utilizing the bioelectric simulator of claim 1 to apply the bioelectric signal(s) to the living cell for a minimum duration of 20 minutes so as to upregulate production of OPG by the living cell.

3. A method of increasing production of osteoprotegerin (OPG) by a cell that produces OPG, the method comprising:
applying at least one bioelectric signal to the cell, wherein the at least one bioelectric signal comprises:
(a) within 15%, 4.0 millivolts, 2,000 Hz, square wave; and/or
(b) from 3 millivolts to 5 millivolts at a frequency range of 1 to 3 MHz, and a duration range of 30 to 40 mW/cm2,
for a minimum of 20 minutes so as to increase production of OPG by the cell, wherein OPG production by the cell is increased.

4. The method according to claim 3, wherein the cell is comprised within a subject.

5. The method according to claim 4, wherein the subject has been diagnosed with a disorder selected from the group consisting of cancer, breast cancer, bone cancer, lung cancer, osteoporosis, and a combination of any thereof.

6. The method according to claim 4, wherein the subject has been diagnosed with multiple myeloma.

7. The method according to claim 4, wherein the at least one bioelectric signal comprises:
within 15%, 4.0 millivolt, 2,000 Hz, square wave for a minimum of 20 minutes.

8. The method according to claim 7, wherein the subject has been diagnosed with multiple myeloma.

9. The method according to claim 4, wherein the at least one bioelectric signal comprises:
from 3 millivolts to 5 millivolts at a frequency range of 1 to 3 MHz, and a duration range of 30 to 40 mW/cm2 for a minimum of 20 minutes.

10. The method according to claim 9, wherein the subject has been diagnosed with multiple myeloma.

11. A method of using a bioelectric stimulator programmed to produce at least one bioelectric signal that stimulate(s) target tissue of a subject in need thereof to increase production of osteoprotegerin (OPG), wherein the bioelectric signal(s) comprise(s):
(a) within 15%, 4.0 millivolts, 2,000 Hz, square wave; and/or
(b) from 3 millivolts to 5 millivolts at a frequency range of 1 to 3 MHz, and a duration range of 30 to 40 mW/cm2 for a minimum of 20 minutes, to treat a disorder in a subject, the method comprising:
delivering the at least one bioelectric signal(s) to tissue of the subject so as to increase production of OPG by the target tissue,
wherein OPG production is increased by the target tissue.

12. The method according to claim 11, wherein the subject has been diagnosed with a disorder selected from the group consisting of cancer, breast cancer, bone cancer, lung cancer, osteoporosis, and a combination of any thereof.

13. The method according to claim 11, wherein the subject has been diagnosed with multiple myeloma.

14. The method according to claim 11, wherein the at least one bioelectric signal to increase production of OPG in the target tissue comprises:
within 15%, 4.0 millivolt, 2,000 Hz, square wave for a minimum of 20 minutes.

15. The method according to claim 14, wherein the subject has been diagnosed with multiple myeloma.

16. The method according to claim 11, wherein the at least one bioelectric signal to increase production of OPG in the target tissue comprises:
from 3 millivolts to 5 millivolts at a frequency range of 1 to 3 MHz, and a duration range of 30 to 40 mW/cm2 for a minimum of 20 minutes.

17. The method according to claim 16, wherein the subject has been diagnosed with multiple myeloma.
